(19) United States
(12) Patent Application Publication
(10) Pub. No.: US 2026/0283825 A1
(43) Pub. Date: Sep. 24, 2026

(54) PRESSURIZED CRIMPED BALLOONS AND STENTS AND METHODS

(71) Applicant: Merit Medical Systems, Inc., South Jordan, UT (US)

(72) Inventors: Mattias Nielsen, North Salt Lake, UT (US); Adam Wilhite, Herriman, UT (US); Jason Wiersdorf, West Jordan, UT (US); Wayne Mower, Bountiful, UT (US); John Hall, Bountiful, UT (US); Yuma Medina, South Jordan, UT (US)

(21) Appl. No.: 19/572,356

(22) Filed: Mar. 19, 2026

Related U.S. Application Data
(60) Provisional application No. 63/775,607, filed on Mar. 21, 2025.

(51) Int. Cl. A61F 2/958; A61M 25/10

(52) U.S. Cl. CPC A61F 2/958; A61M 25/1002; A61F 2002/9583; A61M 2025/1004; A61M 2025/1075; A61M 2025/1086

(57) ABSTRACT

Medical balloons and stents and methods used to retain and expand a stent within a stricture of a bodily passage are disclosed. The balloons include a base material layer and a retention feature to retain the stent on the balloon during deployment to a stricture within a patient's body. The retention feature includes one or more of bulges, pillows, pooches, proud portions, or protuberances of the base material layer that extend radially outward from a nominal circumference of the balloon into interstices of a stent disposed over the balloon. The retention feature is formed by pressurizing the balloon to a target pressure. The balloon and stent are radially crimped while maintaining the target pressure within the balloon by venting fluid from the balloon.

### RELATED CASES

This application claims priority to U.S. Provisional Application No. 63/775,607, filed on Mar. 21, 2025, and titled “PRESSURIZED CRIMPED BALLOONS AND STENTS AND METHODS,” which is hereby incorporated by reference in its entirety.

### TECHNICAL FIELD

The present disclosure relates generally to medical devices to treat a stricture of a bodily passage. More specifically, the present disclosure relates to a balloon to expand a stent placed within the stricture of the bodily passage and methods of producing the balloon.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an embodiment of a tube of an embodiment of a stent expanding balloon.

FIG. 2 is a perspective view of an embodiment of a laminar structure of the tube of FIG. 1.

FIG. 3 is a perspective view of an embodiment of a formed laminar balloon of the laminar structure of FIG. 2.

FIG. 4 is a side cross-sectional view of the formed laminar balloon of FIG. 3.

FIG. 5 is a side view of an embodiment of a stent expanding balloon having a friction feature dispersed over an external surface.

FIG. 6A is a side view of a detailed portion of the stent expanding balloon of FIG. 5 showing an embodiment of the friction feature.

FIG. 6B is a side view of a detailed portion of the stent expanding balloon of FIG. 5 showing another embodiment of the friction feature.

FIG. 7 is a perspective view of another embodiment of a tube of another embodiment of a stent expanding balloon.

FIG. 8 is a perspective view of an embodiment of a formed balloon of the tube of FIG. 7.

FIG. 9A is a side view of the formed balloon of FIG. 8 with longitudinal pleats.

FIG. 9B is a cross-sectional view of the formed balloon of FIG. 9A.

FIG. 10 is a perspective view of the formed balloon of FIG. 8 in a deflated state and with a balloon expandable stent disposed over at least a portion of the formed balloon.

FIG. 11 is a perspective view of the formed balloon of FIG. 10 in an at least partially inflated state.

FIG. 12A is a side view of a stent retention feature of the formed balloon of FIG. 10 in a crimped state.

FIG. 12B is a cross-sectional view of the stent retention feature of the formed balloon of FIG. 12A through section 12B-12B.

FIG. 12C is a side view of the stent retention feature of the section 12C of the formed balloon of FIG. 12A with the balloon expandable stent removed.

FIG. 13 is a side view of the stent retention feature of a portion of the formed balloon of FIG. 12A.

FIG. 14A is a side view of the stent retention feature of a portion of the formed balloon of FIG. 12A in an inflated state.

FIG. 14B is a cross-sectional view of the stent retention feature of the formed balloon of FIG. 14A through section 14B-14B.

FIG. 14C is side view of the stent retention feature of the section 14C of the formed balloon of FIG. 14A with the balloon expandable stent removed.

### DETAILED DESCRIPTION

In certain instances, a stricture may form in a bodily passage, such as a blood vessel, a gastrointestinal tract, etc. The stricture may restrict fluid flow (blood, digestive fluids, etc.) within the bodily passage resulting in morbid or mortal complications. The stricture can be treated by positioning a stent within the stricture using a stent delivery device having a stent expanding balloon. When the stent is positioned, the stent expanding balloon can be inflated causing the stent to expand to open the stricture. In some embodiments, the stent expanding balloon has a slick exterior surface and the stent may not be adequately coupled or secured to the stent expanding balloon during positioning of the stent resulting in premature displacement of the stent from the stent expanding balloon. This may result in improper positioning or expansion of the stent. In other embodiments, the balloon expanding stent inflates from both ends prior to inflating a middle section forming a dog bone shaped inflated balloon. This may cause the stent to accordion from one or both ends toward a middle portion resulting in a shortened stent.

Embodiments herein describe stent expanding balloons and methods having features to increase static stent holding forces. In some embodiments within the scope of this disclosure, the balloons include a first end portion; a second end portion; and a body portion disposed between the first end portion and the second end portion. The body portion can include a stent retention feature configured to increase a static stent holding force. In certain embodiments, the balloons may have a static stent holding force that is greater than five Newtons, greater than 10 Newtons, or greater than 15 Newtons. In some embodiments, the static stent holding force may be between 5 and 20 Newtons, including between 10 and 15 Newtons. The stent retention feature includes a plurality of bulges, pillows, pooches, proud portions, or protuberances that extend radially outward from a nominal circumference of the balloon when the balloon is in a crimped state. In certain embodiments when a stent is disposed over the balloon, the plurality of bulges, pillows, pooches, proud portions, or protuberances extend into interstices of the stent to retain the stent on the balloon.

In another embodiment, the body includes a first material layer defining an interior surface and the stent retention feature includes a second material layer disposed over the first material layer. The second material layer is more compressible than the first material layer. A material of the second material layer has a Shore durometer of between 50 A and 90 A. A thickness of the second material layer ranges between 0.008 millimeter and 0.051 millimeter. The second material layer is one or more of silicone, polyurethane, polyether block amide, and polytetrafluoroethylene. In another embodiment, the second material layer is a sleeve disposed over the first material layer. In another embodiment, the stent retention feature includes a plurality of micro or nano structures extending radially outward from the exterior surface. In one embodiment, the structures include a seta having a projection. In another embodiment, the structures include a nano tube. Each of the structures provide an electrostatic force comprising an attractive van der Waals force of at least 0.4 μN.

In certain embodiments, a method of forming the balloons includes the steps of: extruding a first material to form a tube comprising a first material layer having an interior surface and an exterior surface; blow molding the tube in a mold to form a stent expanding balloon; disposing a balloon expandable stent over the stent expanding balloon; pressurizing the stent expanding balloon; radially crimping the stent expanding balloon and the balloon expandable stent; venting the stent expanding balloon to maintain a crimping pressure of between about 0.25 atmosphere and about 10.0 atmospheres within the stent expanding balloon wherein portions of the stent expanding balloon bulge into interstices of the balloon expandable stent.

In another embodiment, a second material layer is disposed over the exterior surface of the stent expanding balloon, wherein the second material layer is coupled to the exterior surface. In some embodiments, the exterior surface is treated using one or more of dielectric barrier discharge, chemical priming, and mechanical texturing, prior to the step of disposing the second material layer over the exterior surface of the tube. After treatment, the exterior surface has a water contact angle of less than 50 degrees. In another embodiment, the second material layer is disposed over the exterior surface of the tube by co-extruding the second material layer over the first material layer of the tube. The second material layer includes one or more of polyurethane, silicone, and polyether block amide. In another embodiment, the second material layer is disposed over the exterior surface of the tube by dipping the tube into a solvated polymer solution. The solvated polymer solution includes one or more of polyurethane, silicone, and polyether block amide.

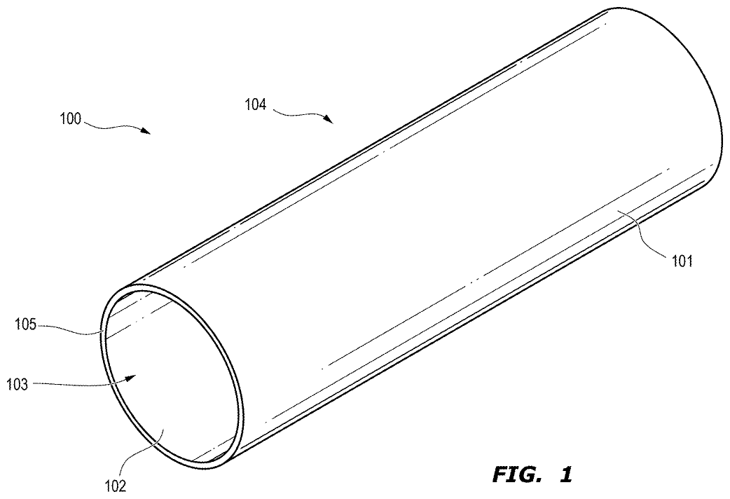

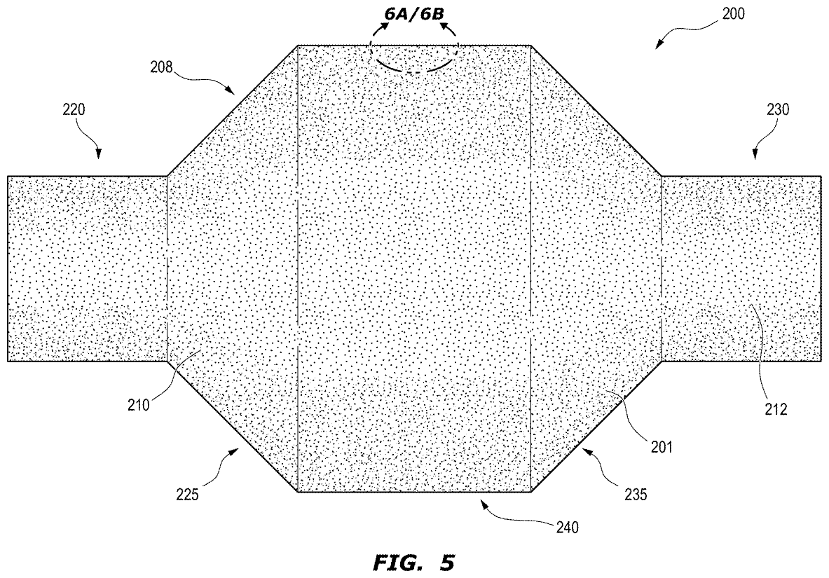

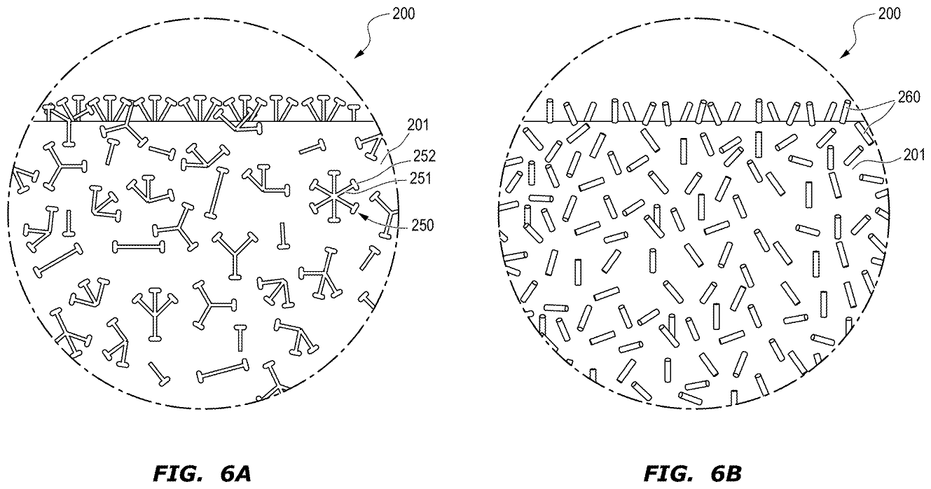

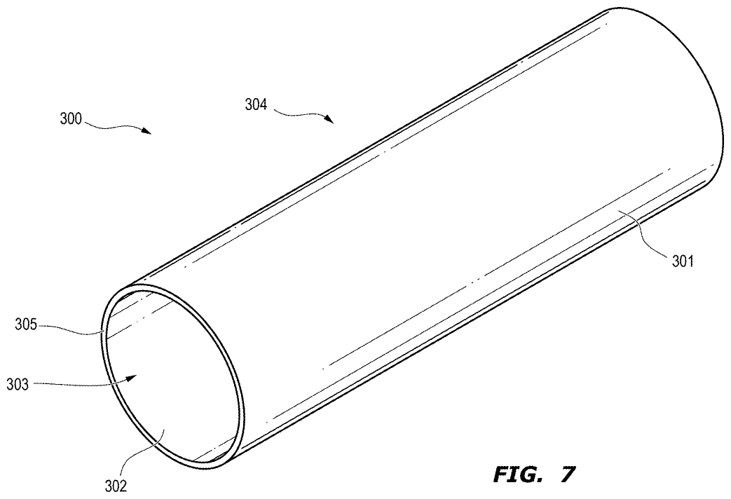

FIG. 1-4 illustrate an embodiment of an embodiment of a stent expanding balloon having a friction feature including a laminar structure. FIG. 5 illustrates an embodiment of a stent expanding balloon having a friction feature dispersed over an external surface. FIG. 6A illustrates a detailed portion of the stent expanding balloon of FIG. 5 showing an embodiment of the friction feature having a plurality of micro or nano sized setae and projections or protrusions. FIG. 6B illustrates a detailed portion of the stent expanding balloon of FIG. 5 showing another embodiment of the friction feature having a plurality of micro or nano sized tubes or columns. FIGS. 7-14C illustrate another embodiment of a stent expanding balloon having a stent retention feature.

In certain views each device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

As illustrated in FIG. 1, a stent expanding balloon 100 includes an extruded tube 104 having a first material layer or base wall 105. The first material layer 105 has an exterior surface 101 and an interior surface 102 defining a bore 103 extending through the length of the tube 104. In the depicted embodiment, the tube 104 has a circular cross-sectional shape. Other cross-sectional shapes are within the scope of this disclosure. An external diameter of the tube 104 may range from about 4 millimeters to about 10 millimeters, including from about 6 millimeter to about 8 millimeters. The first material layer 105 may be formed from any suitable thermoplastic material to provide a compliant, semi-compliant, or non-compliant stent expanding balloon 100. For example, materials such as nylon, polyether block amide, and polyethylene terephthalate may be used. Other materials are within the scope of this disclosure.

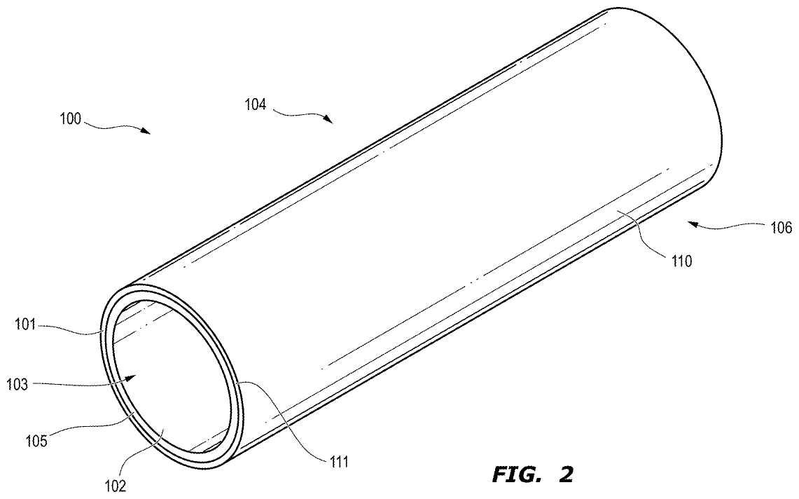

FIG. 2 illustrates the tube 104 of the stent expanding balloon 100 having a friction feature 110 disposed over and coupled to the exterior surface 101. As depicted, the friction feature 110 is a second material layer 111 disposed over and coupled to the exterior surface 101 to form a laminar structure 106. The second material layer 111 may be formed from any suitable material, such as silicone, polyurethane, polyether block amide, and polytetrafluoroethylene. Other materials are within the scope of this disclosure. In some embodiments, second material layer 111 may have a thickness ranging between about 0.0008 millimeter and about 0.051 millimeter. The second material layer 111 may have a first coefficient of friction that is higher or greater than a second coefficient of friction of the first material layer 105. The first coefficient of friction may provide a static stent holding force of at least 5 N to ensure stability of a stent over the stent expanding balloon 100 when the stent is being positioned for deployment within a stricture of a bodily passage, such as a blood vessel, a gastrointestinal tract, etc.

In certain embodiments, the second material layer 111 can be more compressible than the first material layer 105. The second material layer 111 may be formed from any suitable compressible material, such as silicone, polyurethane, and polyether block amide. Other materials are within the scope of this disclosure. The second material layer 111 may have a Shore hardness durometer of from about 50 A to about 90 A, and may be about 70 A.

In some embodiments, the second material layer 111 is disposed over the first material layer 105 and coupled to the exterior surface 101 to form the laminar structure 106 using a co-extrusion manufacturing technique. For example, pellets of the first material are melted in a first extruder. Pellets of the second material are melted in a second extruder. The melted first and second materials flow together with partial mixing at an interface between the materials. The combined materials are extruded through a die under pressure to form the first material layer 105 and the second material layer 111 of the laminar structure 106. In certain embodiments, the first and second materials may have similar melt indices to allow the first and second material layers to melt together to form a melt bond between the first material layer 105 and the second material layer 111.

In another embodiment, the second material layer 111 is disposed over the first material layer 105 and coupled to the exterior surface 101 to form the laminar structure 106 using a dip coating manufacturing technique. For example, the tube 104 is dipped into a solvated solution of the material of the second material layer 111 and a suitable solvent. When the tube 104 is removed from the solvated solution, the exterior surface 101 is coated with the solvated solution. The thickness of the coating may be determined by such factors as viscosity of the solvated solution and withdrawal rate of the tube 104 from the solvated solution. For example, a solvated solution having a high viscosity may form a thicker coating than a solvated solution having a low viscosity and a process having a fast withdrawal rate may form a thinner coat than a process have a slow withdrawal rate. The solvent evaporates from the coating leaving the second material to form the second material layer 111. In some embodiments, the solvent of the solvated solution may partially dissolve or soften the first material layer 105 to provide a solvent bond between the first material layer 105 and the second material layer 111.

In an embodiment, the exterior surface 101 of the tube 104 can be treated (i.e., cleaned or activated) prior to dip coating to enhance bonding between the first material layer 105 and the second material layer 111. The exterior surface 101 may be treated using any suitable technique to provide a water contact angle of less than 50 degrees. For example, the treatment may use dielectric barrier discharge (i.e., plasma or corona) or chemical priming.

In another embodiment, the second material layer 111 may include a sleeve disposed over the tube 104. The sleeve may include an internal surface coated with an adhesive.

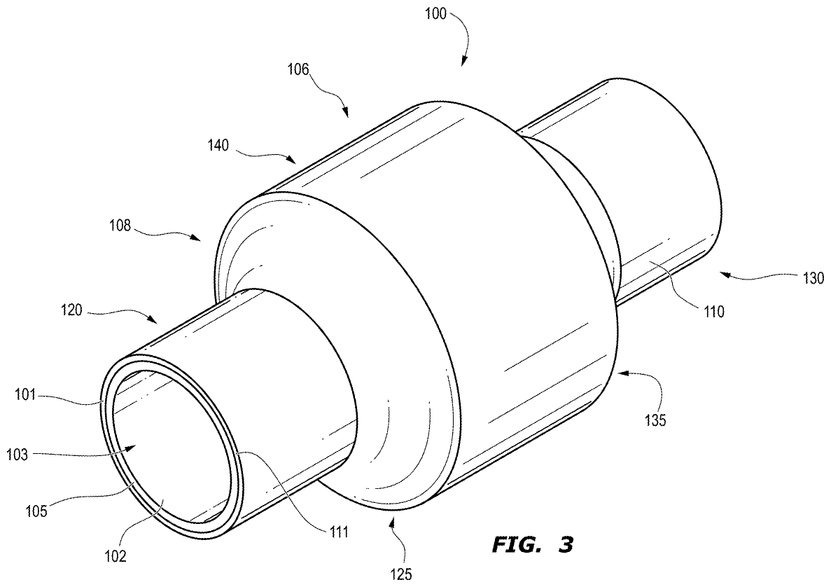

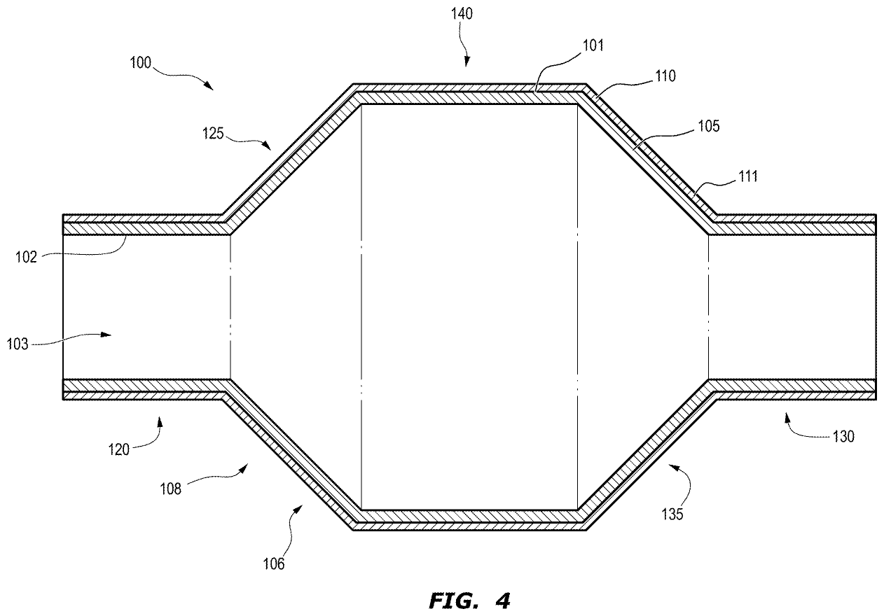

FIGS. 3 and 4 illustrate a formed laminar balloon 108 of the stent expanding balloon 100 formed from the laminar structure 106. As shown in FIG. 3, the formed laminar balloon 108 includes a first end portion 120, a second end portion 130, a middle portion 140 disposed between the first end portion 120 and the second end portion 130, and the friction feature 110. The bore 103 is defined by the interior surface 102 and can extend through the first end portion 120, the middle portion 140, and the second end portion 130 such that the first end portion 120, the middle portion 140 and the second end portion 130 are in fluid communication. An opening is disposed at each end of the bore 103. In some embodiments, the outer diameter of the first end portion 120 is substantially equivalent to the outer diameter of the second end portion 130. In other embodiments, the outer diameter of the first end portion 120 is different (i.e., smaller or larger) than the outer diameter of the second end portion 130.

A first taper region 125 may be disposed between the first end portion 120 and the middle portion 140. A second taper region 135 may be disposed between the second end portion 130 and the middle portion 140. In some embodiments, the length of the first taper region 125 is substantially equivalent to the length of the second taper region 135. In other embodiments, the length of the first taper region 125 is different (i.e., shorter or longer) than the length of the second taper region 135.

In certain embodiments, the formed laminar balloon 108 is formed by disposing the laminar structure 106 within a cavity of a heated mold having a desired stent expanding balloon shape. Air pressure is applied to the bore 103 of the laminar structure 106. Heating of the materials of the first material layer 105 and the second material layer 111 causes the materials to soften. The air pressure causes the first material layer 105 and the second material layer 111 to expand radially outward to conform to a shape of the cavity and to form the formed laminar balloon 108. In some embodiments, a surface of the cavity may be coated with a release agent to prevent sticking of the second material layer 111 to the cavity surface. The first material layer 105 and the second material layer 111 are stretched resulting in a thinning of the layers 105, 111.

FIGS. 5-6B depict an embodiment of a stent expanding balloon 200 that resembles the stent expanding balloon 100 described above in certain respects. Accordingly, like features are designated with like reference numerals, with the leading digit incremented to “2.” For example, the embodiment depicted in FIGS. 5-6B includes a friction feature 210 that may, in some respects, resemble the fracture feature 110 of FIG. 2. Relevant disclosure set forth above regarding similarly identified features thus may not be repeated hereafter. Moreover, specific features of the stent expanding balloon 100 and related components shown in FIGS. 1-4 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the stent expanding balloon 200 and related components depicted in FIGS. 5-6B. Any suitable combination of the features, and variations of the same, described with respect to the stent expanding balloon 100 and related components illustrated in FIGS. 1-4 can be employed with the stent expanding balloon 200 and related components of FIGS. 5-6B, and vice versa. This pattern of disclosure applies equally to further embodiments depicted in subsequent figures and described hereafter, wherein the leading digits may be further incremented.

FIG. 5 illustrates another embodiment of a stent expanding balloon 200. As depicted the stent expanding balloon 200 includes a formed balloon 208 defined by an outer surface 201. The formed balloon 208 includes a first portion 220, a second portion 230, a middle portion 240 disposed between the first portion 220 and a second portion 230, a first taper region 225 disposed between the first portion 220 and the middle portion 240, and a second taper region 235 disposed between the second portion 230 and the middle portion 240. The outer surface 201 includes a friction feature 210 dispersed over the outer surface 201. In another embodiment, the friction feature 210 is dispersed over the outer surface of the middle portion 240, the first taper region 225, and the second taper region 235. In some embodiments, the friction feature 210 is dispersed only over the outer surface 201 of the middle portion 240.

The friction feature 210 includes a plurality of micro or nano sized structures 212 extending from or into the outer surface 201. The structures 212 may be of any suitable shape or form that provides an adequate positive van der Waals force to cumulatively provide a static stent holding force of at least five Newtons. For example, FIG. 6A illustrates an embodiment of micro or nano sized structures 250 dispersed over the exterior surface 201. As depicted, the each of the structures 250 include a seta or hair 251 and a projection or protrusion 252 extending from an end of the seta 251. The projection 252 may be of any suitable shape, such as a spatula or mushroom. Other suitable shapes are within the scope of this disclosure. In some embodiments, the structures 250 include a clump or plurality of setae 251 and projections 252 clustered together. The projection 252 can provide a positive van der Waals force of about 0.4 μN. A plurality of structures 250 dispersed over the outer surface 201 can provide a stent static holding force of at least five Newtons.

FIG. 6B illustrates another embodiment of micro or nano sized structures 260 dispersed over the exterior surface 201. As depicted, the structures 260 include a tube or column extending from the exterior surface 201. Each structure 260 can provide a positive van der Waals force of about 0.4 μN. A plurality of structures 260 dispersed over the outer surface 201 can provide a stent static holding force of at least five Newtons.

In certain embodiments, the friction feature 210 is formed and dispersed over the outer surface 201 during the blow molding process of forming the formed balloon 208. A negative or cavity of the structures 212 can be provided in the cavity of the blow mold such that when the tube 204 is heated and pressurized within the blow mold the material of the first material layer 205 flows into the negative or cavity of the structures 212 to form the structures 212. In another embodiment, the structures 212 are dispersed over the outer surface 201 prior to the blow molding process.

In some embodiments, the friction feature 210 is formed and dispersed over the outer surface 201 by forming the structures 212 on a flexible film or tape and then wrapping the formed balloon 208 with the flexible film or tape. In one embodiment, the structures 212 are formed by roughening the outer surface 201 to increase a surface area of the outer surface 201. The outer surface 201 may be roughened using any suitable technique such as sanding, sand blasting, plasma etching, corona etching, electrochemical etching, or chemical etching. Other roughening techniques are within the scope of this disclosure.

As illustrated in FIG. 7, a stent expanding balloon 300 includes an extruded tube 304 having a first material layer or base wall 305. The first material layer 305 has an exterior surface 301 and an interior surface 302 defining a bore 303 extending through the length of the tube 304. In the depicted embodiment, the tube 304 has a circular cross-sectional shape. Other cross-sectional shapes are within the scope of this disclosure.

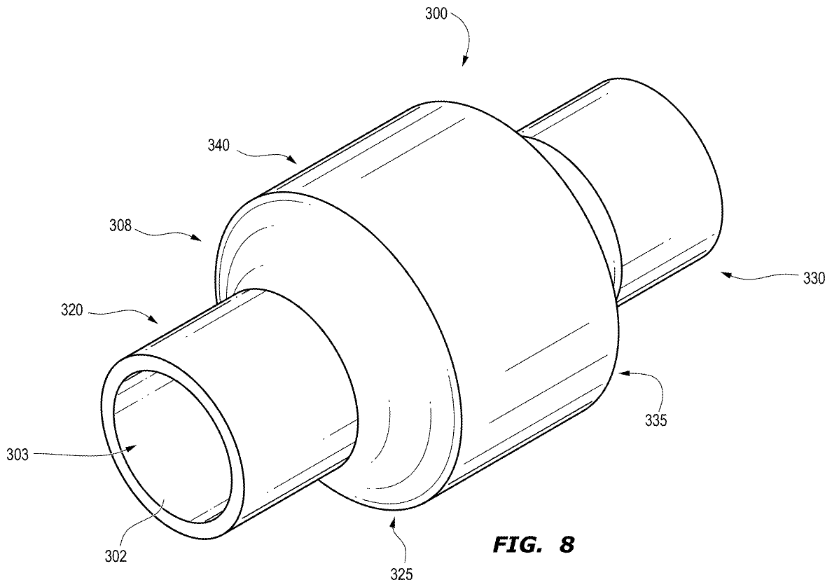

FIG. 8 illustrates a formed balloon 308 of the stent expanding balloon 300 formed from the tube 304. As shown in FIG. 8, the formed balloon 308 includes a first end portion 320, a second end portion 330, and a middle portion 340 disposed between the first end portion 320 and the second end portion 330. The bore 303 is defined by the interior surface 302 and can extend through the first end portion 320, the middle portion 340, and the second end portion 330 such that the first end portion 320, the middle portion 340 and the second end portion 330 are in fluid communication. An opening is disposed at each end of the bore 303. A first taper region 325 may be disposed between the first end portion 320 and the middle portion 340. A second taper region 335 may be disposed between the second end portion 330 and the middle portion 340.

In certain embodiments, the formed balloon 308 is formed by disposing the tube 304 within a cavity of a heated mold having a desired stent expanding balloon shape. Air pressure is applied to the bore 303 of the tube 304. Heating of the materials of the first material layer 305 causes the material to soften. The air pressure causes the first material layer 305 to expand radially outward to conform to a shape of the cavity and to form the formed balloon 308. In some embodiments, a surface of the cavity may be coated with a release agent to prevent sticking of the first material layer 305 to the cavity surface.

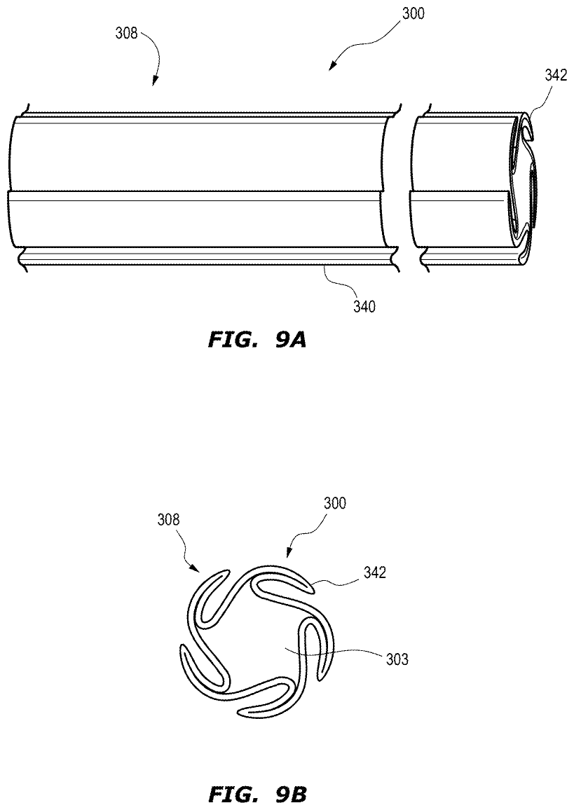

In certain embodiments, the stent expanding balloon 300 includes longitudinal pleats 342 disposed circumferentially along the middle portion 340 between the first end portion 320 and the second end portion 330 as shown in FIGS. 9A and 9B. The pleats 342 can allow the stent expanding balloon 300 to be wrapped around a stent delivery catheter such that the wrapped stent expanding balloon 300 has a smaller diameter than the stent expanding balloon 300 without the pleats 342. The number of pleats 342 may be one, two, three, four, five, or more equally distributed about the circumference of the middle portion 340. The pleats 342 can be formed by disposing an inflated formed balloon 308 into a heated balloon pleating die and then applying a vacuum to the formed balloon 308.

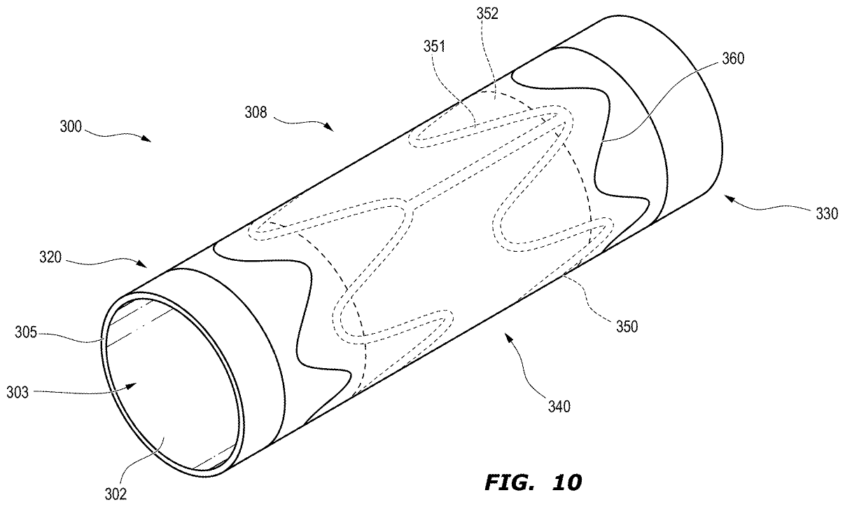

As shown in FIG. 10, a balloon expandable stent 350 is disposed over the formed balloon 308 when the formed balloon 308 is deflated. The balloon expandable stent 350 may be disposed over one or more of the first end portion 320, the second end portion 330, and the middle portion 340. In another embodiment, the balloon expandable stent 350 may be disposed over the middle portion 340. The balloon expandable stent 350 includes struts 351 and interstices 352 disposed between the struts 351. In certain embodiments, the balloon expandable stent 350 may be formed from a metal tube that is laser cut to form the struts 351 and interstices 352. In other embodiments, the balloon expandable stent 350 can be formed from metal wire that is braided or woven to form the struts 351 and the interstices 352. The struts 351 may be configured in any suitable pattern to prevent collapse of the balloon expandable stent 350 when disposed within a stricture of a body passage and to permit radial crimping to facilitate delivery of the balloon expandable stent to the stricture. As illustrated in FIG. 10, a cover 360 may be disposed over the balloon expanding stent 350. The cover 360 can be formed from any suitable material, such as polytetrafluoroethylene, polyurethane, silicone, etc. The cover 360 may cover all or any portion of the balloon expandable stent 350.

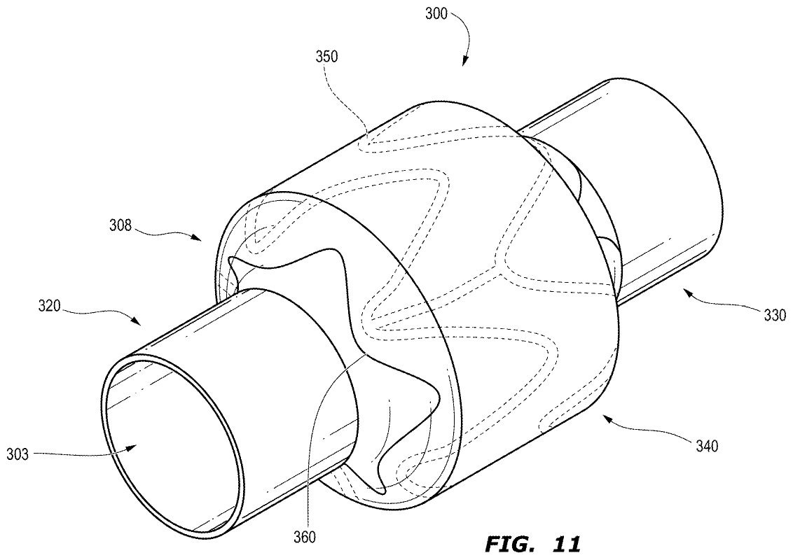

FIG. 11 shows the formed balloon 308 in a pressurized and inflated state with the balloon expandable stent 350 in an expanded state prior to radial crimping. The formed balloon 308 can be pressurized to from about 0.25 atmosphere to about 10.0 atmospheres with any suitable fluid, such as air or water. When the formed balloon 308 is pressurized and inflated, the formed balloon 308 and the balloon expandable stent 350 may be radially pressure crimped to reduce the outer diameter of the formed balloon 308 and the balloon expandable stent 350. The reduced outer diameter may facilitate placement of the stent expanding balloon 300 and the balloon expandable stent 350 within a stent delivery sheath. Additionally, the radial pressure crimping can form a stent retention feature to retain the balloon expandable stent 350 on the stent expanding balloon 300 during a stent deployment procedure.

During radial pressure crimping, the pressure within the formed balloon 308 may be maintained at a target crimping pressure of about 0.25 atmosphere to about 10.0 atmospheres, about 0.5 atmosphere to about 8.0 atmospheres, and at about 1.0 atmosphere. The target crimping pressure can be maintained by venting the fluid from the formed balloon 308 as the outer diameter of the formed balloon 308 decreases. The venting can be controlled using any suitable technique. For example, the venting is controlled by a pressure control valve coupled in fluid communication with the bore 303 and a controller coupled to a pressure sensor in fluid communication with the bore 303. The pressure control valve can increase or decrease fluid flow from the formed balloon 308 through the pressure control valve in response to the crimping pressure sensed by the pressure sensor. For example, when a pressure within the formed balloon 308 exceeds a target crimping pressure of 1.0 atmosphere, the controller can send a signal to the pressure control valve to open an additional amount to permit more fluid to flow from the formed balloon 308 through the pressure control valve resulting in a pressure decrease within the formed balloon 308 to the target crimping pressure.

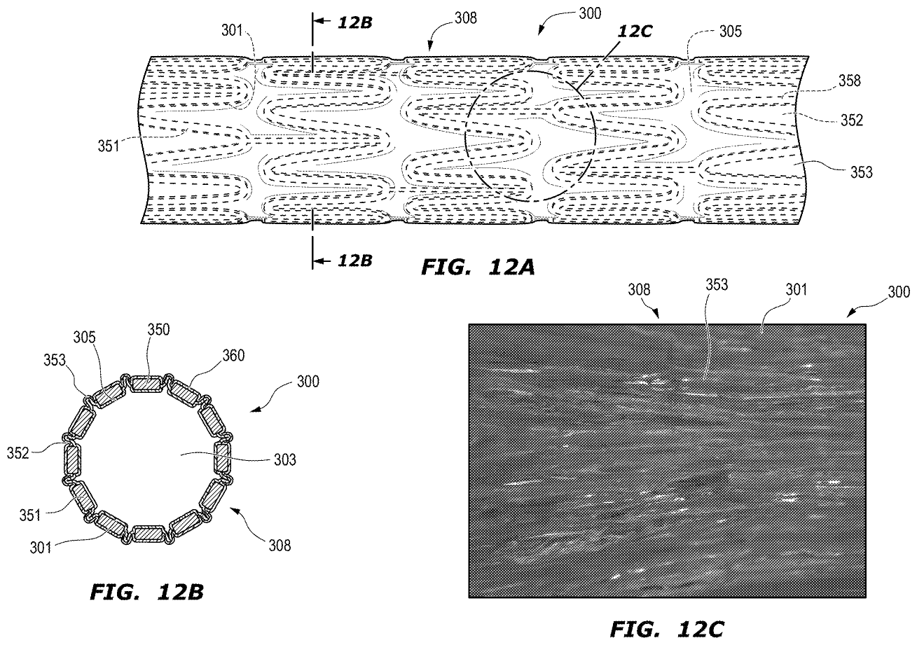

FIGS. 12A, 12B, and 12C illustrate the formed balloon 308 of the stent expanding balloon 300 and the balloon expandable stent 350 in crimped states. As shown, the formed balloon 308 includes a stent retention feature 353 that includes bulges, pillows, pooches, proud portions, or protuberances extending radially outward from a nominal circumference of the formed balloon 308 into the interstices 352 of the balloon expandable stent 350. The bulges, pillows, pooches, proud portions, or protuberances of the stent retention feature 353 are formed of portions of the first material layer 305. During pressure crimping, the first material layer 305 is forced against the fluid within the bore 303 of the formed balloon 308. The fluid is under pressure and pushes against the first material layer 305 causing the first material layer 305 to be displaced radially outward into and through the interstices 352 of the balloon expandable stent 350 to form the stent retention feature 353.

As shown in FIG. 12B, the stent retention feature 353 can extend into or through the interstices 352 to engage with the struts 351 and prevent the balloon expandable stent 350 from axial displacement from the stent expanding balloon 300 during placement of the balloon expandable stent 350 within the stricture of the body passage. This height may be greater than or less than the thickness of the struts 351. FIG. 12C shows a portion of the crimped formed balloon 308 with the balloon expandable stent 350 removed. The bulges, pillows, pooches, proud portions, or protuberances of the stent retention feature 353 are shown dispersed over the exterior surface 301. The stent retention feature 353 may provide a stent holding force between the stent expanding balloon 300 and the balloon expandable stent 350. In embodiments that include the cover 360, the cover 360 can be displaced radially outward by the stent retention feature 353, as shown in FIG. 12B.

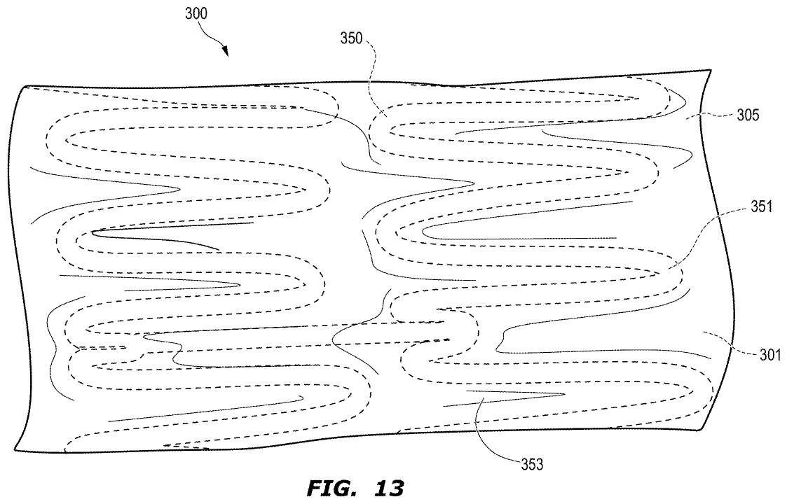

In one embodiment, as shown in FIG. 13, the stent expanding balloon 300 and balloon expandable stent 350, following pressure crimping, may be passed through a ring gauge having a desired diameter to provide a stent expanding balloon 300 having a constant diameter. The ring gauge can flatten the bulges, pillows, pooches, proud portions, or protuberances of the stent retention feature 353 wherein their height is substantially equivalent to or less than the thickness of the struts 351. The flattened stent retention feature 353 can engage the struts 351 to prevent axial displacement of the balloon expandable stent 350 from the stent expanding balloon 300 during placement of the balloon expandable stent 350 within the stricture.

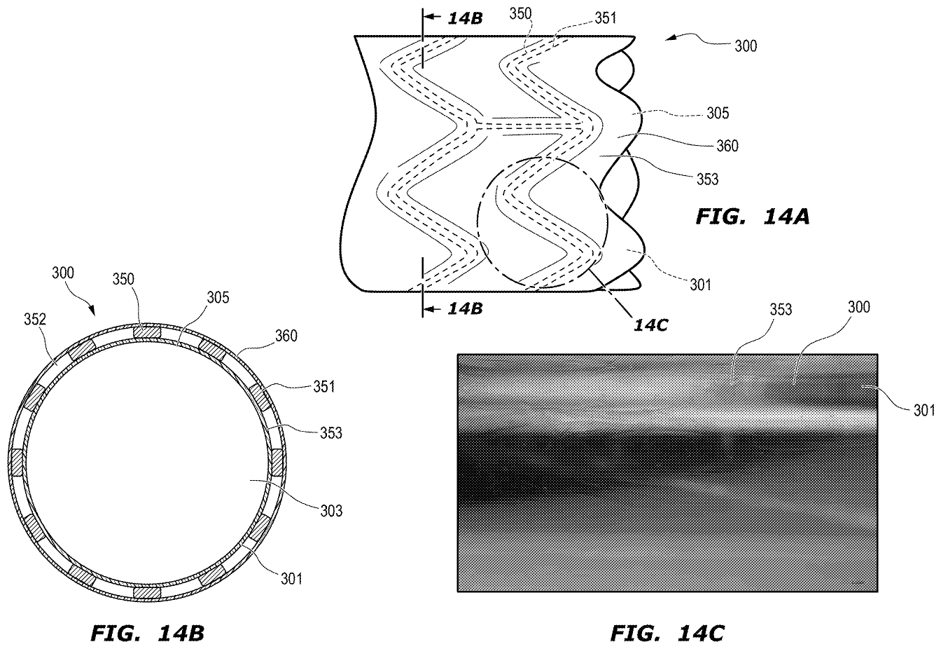

FIGS. 14A, 14B, 14C illustrate the stent expanding balloon 300 and the balloon expandable stent 350 in an expanded state such as when the balloon expandable stent 350 is deployed within a stricture. As shown in FIGS. 14A and 14B, the stent expanding balloon 300 is pressurized to a pressure to expand the balloon expandable stent 350 to an expanded diameter and to dilate the stricture to a desired diameter. When pressurized, the first material layer 305 can be circumferentially tightened or stretched causing the height of the bulges, pillows, pooches, proud portions, or protuberances of the stent retention feature 353 to be substantially zero millimeter. This releases contact of the stent retention feature 353 from the struts 351 allowing the stent expanding balloon 300 to be removed from the balloon expandable stent 350 while leaving the balloon expandable stent 350 at the desired position within the stricture. FIG. 14C illustrates the exterior surface 301 being substantially smooth following pressurization of the stent expanding balloon 300. In other words, the bulges, pillows, pooches, proud portions, or protuberances of the stent retention feature 353 are circumferentially stretched resulting in the smooth exterior surface 301.

In certain embodiments, the stent retention feature 353 can be combined with the previously described friction features 110 and/or 210 as previously described. This combination may provide an increased stent holding force relative the stent retention force provided by the individual features 110, 210, and 353.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of manufacturing a balloon expandable stent system may include one or more of the following steps: disposing a balloon expandable stent around at least a portion of a body portion of a stent expanding balloon, wherein the balloon expandable stent comprises a plurality of struts and a plurality of interstices; inflating the stent expanding balloon to a target crimping pressure; applying a radial crimping force to the inflated stent expanding balloon and the balloon expandable stent to reduce the outer diameters of the stent expanding balloon and the balloon expandable stent to a crimped state; venting the stent expanding balloon to maintain the target crimping pressure within the stent expanding balloon; and displacing portions of a base layer of the stent expanding balloon into the plurality of interstices of the balloon expandable stent. Other steps are also contemplated.

In the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

It will be appreciated that various features are sometimes grouped together in a single embodiment, figure, or description thereof for the purpose of streamlining the disclosure. Many of these features may be used alone and/or in combination with one another.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

“Fluid” is used in its broadest sense, to refer to any fluid, including both liquids and gases as well as solutions, compounds, suspensions, etc., which generally behave as fluids.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially equivalent” is recited with respect to a feature, it is understood that in further embodiments, the feature can have a precisely equivalent configuration.

The terms “a” and “an” can be described as one, but not limited to one. For example, although the disclosure may recite a structure having “a seta,” the disclosure also contemplates that the housing can have two or more setae.

Unless otherwise stated, all ranges include both endpoints and all numbers between the endpoints.

Recitation in the claims of the term “first” with respect to a feature or element does not necessarily imply the existence of a second or additional such feature or element.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A medical balloon, comprising:
a first material layer defining:
a first end portion;
a second end portion;
a body portion disposed between the first end portion and the second end portion; and
a stent retention feature comprising a portion of the first material extending radially outward from a nominal circumference of the medical balloon when the medical balloon is in a crimped state.

2. The medical balloon of claim 1, wherein the stent retention feature is configured to provide a stent holding force of greater than five Newtons when a balloon expandable stent is disposed around at least the body portion in the crimped state.

3. The medical balloon of claim 1, wherein the stent retention feature comprises one or more of a bulge, a pillow, a pooch, a proud portion, and a protuberance.

4. The medical balloon of claim 3, wherein a height of the stent retention feature extends above the nominal circumference of the balloon.

5. The medical balloon of claim 1, wherein the body portion comprises a plurality of longitudinal pleats.

6. The medical balloon of claim 3,
wherein the first material layer comprises an exterior surface,
wherein the stent retention feature further comprises a second material layer disposed over and coupled to the exterior surface, and
wherein the second material layer is more compressible than the first material layer.

7. The medical balloon of claim 6, wherein the second material layer comprises a material having a Shore durometer of between 50 A and 90 A.

8. The medical balloon of claim 6, wherein the second material layer comprises a material having a Shore durometer of 70 A.

9. The medical balloon of claim 6, wherein a thickness of the second material layer ranges between 0.008 millimeter and 0.051 millimeter.

10. The medical balloon of claim 6, wherein the second material layer comprises one or more of silicone, polyurethane, polyether block amide, and polytetrafluoroethylene.

11. The medical balloon of claim 6, wherein the second material layer comprises a sleeve disposed over the first material layer.

12. The medical balloon of claim 1, wherein the stent retention feature further comprises a plurality of micro or nano sized structures extending radially outward from an exterior surface of the medical balloon.

13. The medical balloon of claim 12, wherein at least one of the plurality of structures comprises a seta comprising a projection disposed at a free end of the seta.

14. The medical balloon of claim 12, wherein at least one of the plurality of structures comprises a nano tube.

15. The medical balloon of claim 12, wherein at least one the of plurality of structures provides an electrostatic force comprising an attractive van der Waals force of at least 0.4 μN.

16. A balloon expandable stent system, comprising:
a stent expanding balloon comprising:
a first material layer defining:
a first end portion;
a second end portion; and
a body portion disposed between the first end portion and the second end portion; and
a stent retention feature; and
a balloon expandable stent disposed around at least a portion of the body portion, wherein the balloon expandable stent comprises a plurality of struts and a plurality of interstices disposed between adjacent struts of the plurality of struts,
wherein the stent retention feature extends radially outward from a nominal circumference of the body portion into the plurality of interstices when the stent expanding balloon and balloon expandable stent are in a crimped state.

17. A method of manufacturing a balloon expandable stent system, comprising:
disposing a balloon expandable stent around at least a portion of a body portion of a stent expanding balloon, wherein the balloon expandable stent comprises a plurality of struts and a plurality of interstices;
inflating the stent expanding balloon to a target crimping pressure;
applying a radial crimping force to the inflated stent expanding balloon and the balloon expandable stent to reduce the outer diameters of the stent expanding balloon and the balloon expandable stent to a crimped state;
venting the stent expanding balloon to maintain the target crimping pressure within the stent expanding balloon; and
displacing portions of a base layer of the stent expanding balloon into the plurality of interstices of the balloon expandable stent.

18. The method of claim 17, wherein the target crimping pressure ranges between 0.25 atmosphere and 10.0 atmospheres.

19. The method of claim 17, further comprising passing the stent expanding balloon and balloon expandable stent in the crimped state through a ring gauge.

20. The method of claim 17, further comprising folding the stent expanding balloon to form a plurality of longitudinal pleats.
